(19) United States
(12) Patent Application Publication
(10) Pub. No.: US 2021/0090196 A1
(43) Pub. Date: Mar. 25, 2021

(54) MECHANISM TO SUGGEST CAR SERVICE BASED ON TRANSPORTATION ASSISTANCE NEEDED

(71) Applicant: INTERNATIONAL BUSINESS MACHINES CORPORATION, Armonk, NY (US)

(72) Inventors: Bhargav Cheenepalli, Charlotte, NC (US); David Blake Werts, Charlotte, NC (US); Stephen Douglas Bowman, Monroe, NC (US); Kristin E. McNeil, Charlotte, NC (US)

(21) Appl. No.: 16/580,520

(22) Filed: Sep. 24, 2019

(51) Int. Cl. G06Q 50/30; G06Q 10/02; G10L 13/10; G06Q 10/06; G06N 3/04; G16H 10/60; G06F 16/33

(52) U.S. Cl. CPC G06Q 50/30; G06Q 10/02; G10L 13/10; G06F 16/3344; G06N 3/04; G16H 10/60; G06Q 10/06311

(57) ABSTRACT

A method, apparatus, and non-transitory computer readable medium for transportation services using text analytics are described. The method, apparatus, and non-transitory computer readable medium may provide for inputting a text corpus comprising patient medical information, performing text analytics on the text corpus, determining if the patient has a need for transportation assistance based on the performed text analytics, and notifying a transportation service of the need for transportation assistance based on the determination.

### BACKGROUND

The following relates generally to providing transportation services, and more specifically to providing medical transportation services based on patient need.

In many cases, access to health care depends on the availability of reliable transportation. However, access to reliable transportation is not readily available to a substantial portion of the population. This can result in missed medical appointments or lack of care, and can act as a major barrier to providing proper medical treatment.

Various systems and methods are available that advise patients on transportation options or travel directions, but existing systems do not otherwise help patients obtain transportation. Therefore, it would be desirable for an automated transportation services to schedule transportation services.

### SUMMARY

A method, apparatus, and non-transitory computer readable medium for transportation services using text analytics are described. The described systems and methods may input a text corpus comprising patient medical information, perform text analytics on the text corpus, determine if the patient has a need for transportation assistance based on the performed text analytics, and notify a transportation service of the need for transportation assistance based on the determination.

### BRIEF DESCRIPTION OF THE DRAWINGS

FIG. 1 shows an example of a transportation assistance system in accordance with aspects of the present disclosure.

FIG. 2 shows an example of a server in accordance with aspects of the present disclosure.

FIGS. 3 through 6 show examples of a process for notifying a transportation service based on text analytics in accordance with aspects of the present disclosure.

### DETAILED DESCRIPTION

A fair portion of the population, especially low-income families residing in urban, suburban or rural areas, often miss their medical appointments due to lack of reliable transportation. This acts as a major barrier to medical treatment and negatively impacts health of numerous patients. Further, doctor's appointments fill up fast due to high demand, and average wait times for patients are typically months. This can adversely affect patient health.

Some systems use spoken language to interact with patients and advise them on transportation options or travel directions, with additional assistance based on financial considerations. These systems may include public and/or private transportation information including transportation by air, transportation timetables and availability information for the recommendation of transportation plans, travel agent information, or travel network related information. Although these systems provide patients with assistance scheduling transportation services or best transportation routes, they may not provide automatic real-time identification of a patient needing assistance for transportation.

Thus, embodiments of the present disclosure describe systems and methods that provide real-time analysis and assistance for patients in need of transportation. In some embodiments, methods for scheduling transportation services include automatic identification that a patient needs assistance with scheduling transportation service based on text analytics of a corpus of text inducing patient medical information. For example, embodiments of the present disclosure may include determining if the patient needs assistance with transportation based on the text analytics and notifying a transportation service of the need for transportation assistance based on the determination. Embodiments of the present disclosure provide retrieving patient's preferences and/or appointment data from a structured or unstructured source. Once the patient preference data is retrieved, a transportation service may be scheduled based on the patient's preference data.

Embodiments of the present disclosure may directly or indirectly reduce non-operational time spent by doctors. This may result in the reduction of costs associated with missed appointments, and may also enable doctors to provide care to more patients.

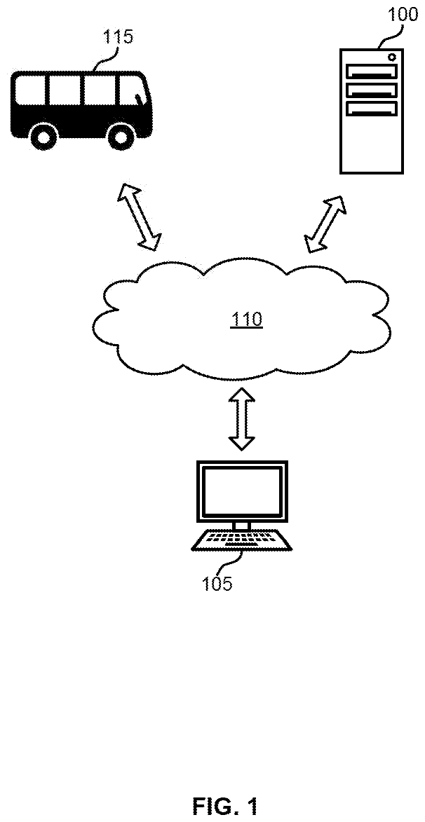

FIG. 1 shows an example of a transportation assistance system in accordance with aspects of the present disclosure. The example shown includes server 100, terminal 105, network 110, and transportation service 115. The terminal 105 may be used by a patient to input information, after which the server 105 may analyze the input information, along with other patient information and transportation information, to schedule transportation services 115 for the patient.

Server 100 may be a computing device, such as a general hardware platform server configured to support computer applications, mobile applications, software, and the like executed on terminal 105. Server 100 may be configured to receive and transmit information over network 110. Server 100 may include physical computing devices residing at a particular location or may be deployed in a cloud computing network environment. Server 100 may include any combination of one or more computer-usable or computer-readable media. For example, server 100 may include a computer-readable medium including one or more of a hard disk, a random access memory (RAM) device, a read-only memory (ROM) device, an erasable programmable read-only memory (EPROM or Flash memory) device, a portable compact disc read-only memory (CDROM), an optical storage device, a magnetic storage device, etc.

Terminal 105 may be a smart phone, desktop computer, tablet computer, laptop computer, wearable computer, personal data assistant, or any other type of device with a hardware processor that is configured to process instructions and connect to network 110, one or more portions of network 110.

Network 110 may be a wired or wireless network such as the Internet, an intranet, a cellular network, a LAN, a WAN, or another type of network. Network 110 may be a combination of multiple different kinds of wired or wireless networks.

In some examples, a patient may use terminal 105 connected to network 110 to input medical text corpus to the transportation assistance system. In some cases, the patient may provide authorization or access so that the transportation assistance system may access patient medical information. Terminal 110 may send the input medical text corpus (and/or authorization) to server 100. Server 100 may process the input medical text corpus (e.g., by performing text analytics) to determine if the patient has a need for transportation assistance. Server 100 may also retrieve patient appointment data and patient preference data. Then, server 100 may automatically schedule transportation service based on the determined needs of the patient, the appointment data, and the patient preference data.

Server 100 may send information to terminal 105 (via network 110) concerning the transportation service that has been scheduled. The patient may view information regarding the transportation service on terminal 105. In some cases, the server may also configure one or more notifications, such as short message service (SMS) notifications for the patient.

A transportation service 115 may include a transportation service operated by a medical facility (i.e., a bus, car, aircraft), a public transportation service (e.g., a bus or train), a ride hailing service, or any other suitable transportation service. In some cases, the transportation service may be scheduled based on special transportation needs of the user. For example, the system may determine that a patient is in a wheelchair, then identify and schedule a transportation service that is capable of providing service to such a patient.

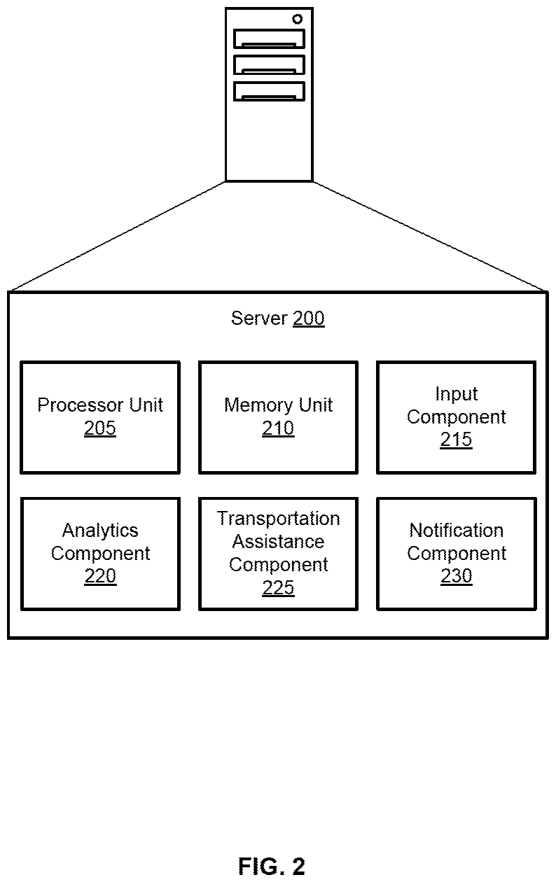

FIG. 2 shows an example of a server 200 in accordance with aspects of the present disclosure. Server 200 may include processor unit 205, memory unit 210, input component 215, analytics component 220, transportation assistance component 225, and notification component 230.

Processor unit 205 may include an intelligent hardware device, (e.g., a general-purpose processing component, a digital signal processor (DSP), a central processing unit (CPU), a graphics processing unit (GPU), a microcontroller, an application specific integrated circuit (ASIC), a field programmable gate array (FPGA), a programmable logic device, a discrete gate or transistor logic component, a discrete hardware component, or any combination thereof). In some cases, processor unit 205 may be configured to operate a memory array using a memory controller. In other cases, a memory controller may be integrated into processor unit 205. Processor unit 205 may be configured to execute computer-readable instructions stored in a memory to perform various functions. In some examples, processor unit 205 may include special purpose components for modem processing, baseband processing, digital signal processing, or transmission processing. In some examples, processor unit 205 may comprise a system-on-a-chip. In some cases, processor unit 205 may be configured to execute computer-readable instructions stored in memory unit 210 to schedule transportation service.

Memory unit 210 may store information for various programs and applications on a computing device. For example, the storage may include data for running an operating system. Memory unit 210 may include both volatile memory and non-volatile memory. Volatile memory may random access memory (RAM), and non-volatile memory may include read-only memory (ROM), flash memory, electrically erasable programmable read-only memory (EEPROM), digital tape, a hard disk drive (HDD), and a solid state drive (SSD). Memory unit 210 may include any combination of readable and/or writable volatile memories and/or non-volatile memories, along with other possible storage devices. In some examples, memory unit 210 may store instructions and data used by server 200 to schedule transportation service.

Input component 215 may input a text corpus comprising patient medical information. In some cases, the text corpus includes text or audio input obtained from a patient accessing a transportation assistance system via a computer or mobile device. For example, the patient could talk into a microphone, or provide information via a form, or provide medical records.

Input component 215 may also retrieve patient data such as patient appointment data from a structured or unstructured data field or database. Input component 215 may also retrieve a patient's preference data to automatically schedule transportation. Input component 215 may also retrieve patient availability information.

Input component 215 may also access transport availability data of a transportation service. For example, input component 215 may access a database of information related to vehicle availability, driver availability, or service availability (i.e., transportation service scheduling information). This information may be provided to the user, or used to automatically schedule a transportation service at an appropriate time.

Analytics component 220 may perform text analytics on the text corpus. In some examples, the text analytics is performed using natural language processing (NLP). In some examples, the NLP is performed using dictionary and rule based processing. In some examples, the NLP is performed using machine learning. In some examples, the machine learning includes a Convolutional Neural Network (CNN), an Unsupervised Pretrained Network (UPN), a Recurrent Neural Network (RNN), a Long Short-Term Memory (LSTM) architecture, a Recursive Neural Network or any other suitable machine learning architecture.

That is, in some embodiments, the analytics component 220 may utilize an artificial neural network (ANN) for performing text analytics. An ANN may be a hardware or a software component that includes a number of connected nodes (a.k.a., artificial neurons), which may be seen as loosely corresponding to the neurons in a human brain. Each connection, or edge, may transmit a signal from one node to another (like the physical synapses in a brain). When a node receives a signal, it can process the signal and then transmit the processed signal to other connected nodes. In some cases, the signals between nodes comprise real numbers, and the output of each node may be computed by a function of the sum of its inputs. Each node and edge may be associated with one or more node weights that determine how the signal is processed and transmitted.

During the training process, these weights may be adjusted to improve the accuracy of the result (i.e., by minimizing a loss function which corresponds in some way to the difference between the current result and the target result). The weight of an edge may increase or decrease the strength of the signal transmitted between nodes. In some cases, nodes may have a threshold below which a signal is not transmitted at all. The nodes may also be aggregated into layers. Different layers may perform different transformations on their inputs. The initial layer may be known as the input layer and the last layer may be known as the output layer. In some cases, signals may traverse certain layers multiple times.

For example, training data may include unstructured text documents along with target input including determination of whether a patient needs a form of transportation. A neural network may also be trained to generate an output vector that includes information such as special transportation needs, scheduling restrictions, and location information in addition to identifying the transportation need.

In some examples, the transportation need may depend on factors such as whether a patient has their own transportation, their location, their medical status, the timing of the medical appointment, or any other information that would help identify a transportation need.

In some cases, unstructured text may be processed using a NLP text processing system before using machine learning to identify the transportation need. For example, unstructured text may be converted to a structured format. The structured information may then be used as the input for the machine learning model. In other examples, unstructured text may be used directly as the input for the machine learning model.

Transportation assistance component 225 may determine if the patient has a need for transportation assistance based on the text analytics. Notification component 230 may notify a transportation service of the need for transportation assistance based on the determination. For example, the notification component 230 may provide notification to the patient, as well as to the provider of the transportation.

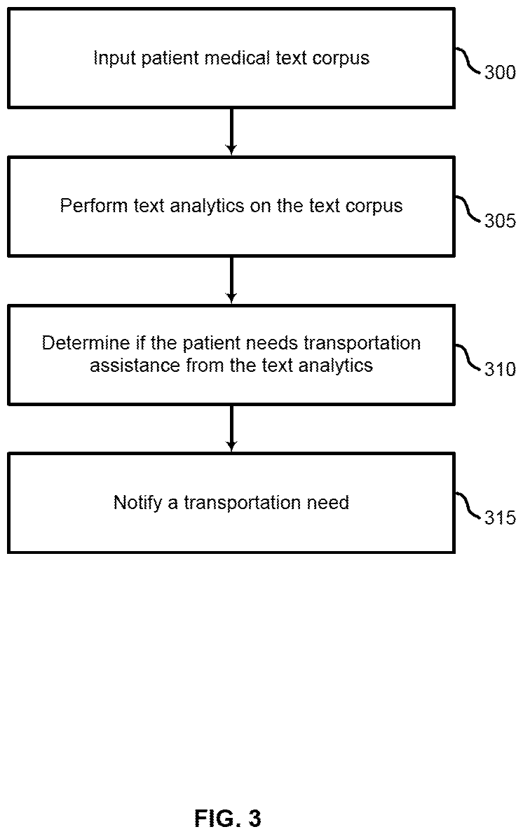

FIG. 3 shows an example of a process for notifying a transportation service based on text analytics in accordance with aspects of the present disclosure. In some examples, these operations may be performed by a system including a processor executing a set of codes to control functional elements of an apparatus. Additionally or alternatively, the processes may be performed using special-purpose hardware. Generally, these operations may be performed according to the methods and processes described in accordance with aspects of the present disclosure. For example, the operations may be composed of various substeps, or may be performed in conjunction with other operations described herein.

At step 300, the system inputs a text corpus comprising patient medical information. In some cases, the operations of this step may refer to, or be performed by, an input component as described with reference to FIG. 2.

For example, a patient may access the system via a user terminal (i.e., a computer or mobile device). The patient may provide information, audio recordings, documents, or forms. In some examples, the input text corpus is a collection of medical documents related to the patient, which may be retrieved from an internal database or via a third party database. In some cases, the text corpus includes electronic medical records. The patient may provide access to the medical records via the user terminal.

At step 305, the system performs text analytics on the text corpus. In some cases, the operations of this step may refer to, or be performed by, an analytics component as described with reference to FIG. 2. In some examples, the text analytics is performed on the text corpus comprising patient medical information. The text analytics may be performed using NLP. In some examples, the NLP is performed using dictionary and rule based processing. In some examples, the NLP is performed using machine learning. In some examples, the machine learning may involve use of a CNN, UPN, RNN, LSTM, Recursive Neural Network, or any other suitable machine learning architecture. In some examples, a need for transportation is identified at step 305 based on the text analytics.

In some examples, the NLP process may include the steps of text pre-processing (i.e., converting the text to a standard format), text parsing (i.e., breaking the text down into portions such as individual words or phrases), text representation (i.e. identifying relevant features of the text), and model application (i.e., applying a model that connects the relevant features to target features such as transportation needs and preferences).

At step 310, the system determines if the patient has a need for transportation assistance based on the performed text analytics. In some cases, the operations of this step may refer to, or be performed by, a transportation assistance component as described with reference to FIG. 2. In some cases, the step 310 may include an engine interpreting annotation output of the text analytics. Based on the interpretation the engine may determine if the patient has a need for transportation assistance. For example, if a patient document in an electronic medical record states “patient needs driving assistance”, “patient is prohibited from driving”, “patient is unable to drive”, or similar, system may determine that the patient needs transportation assistance. In some cases, the need for transportation assistance may be identified during the course of the text analytics (i.e., using a neural network) rather than during a distinct temporal stage.

At step 315, the system notifies a transportation need. In some cases, the operations of this step may refer to, or be performed by, a notification component as described with reference to FIG. 2. In some examples, the method of the present disclosure may use the determination at step 310 to notify the transportation need. In an example, once it is determined that a patient needs transportation assistance, a hospital transportation service may be scheduled. In another example, a private taxi or ride-hailing service may be notified.

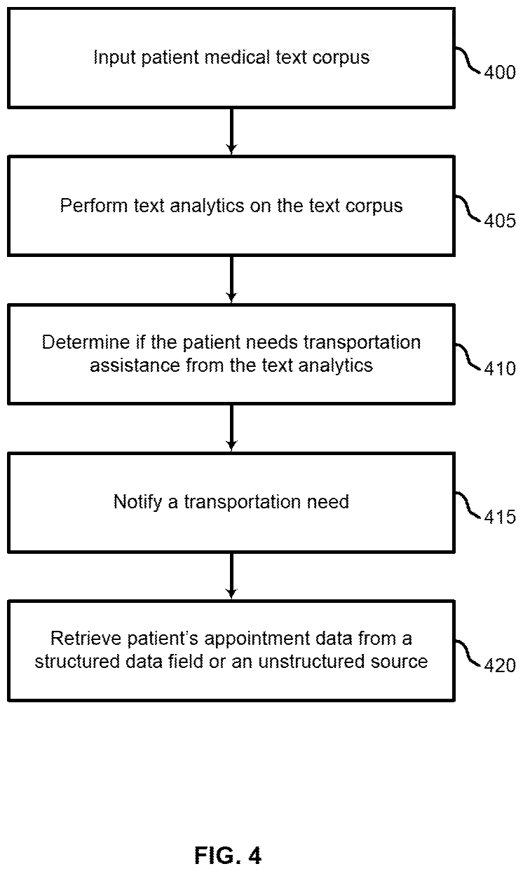

FIG. 4 shows an example of a process for notifying a transportation service based on text analytics in accordance with aspects of the present disclosure. In some examples, these operations may be performed by a system including a processor executing a set of codes to control functional elements of an apparatus. Additionally or alternatively, the processes may be performed using special-purpose hardware. Generally, these operations may be performed according to the methods and processes described in accordance with aspects of the present disclosure. For example, the operations may be composed of various substeps, or may be performed in conjunction with other operations described herein. Steps 400 through 415 may be similar to the corresponding steps for FIG. 3, and further description of these steps is omitted.

At step 400, the system inputs a text corpus comprising patient medical information. In some cases, the operations of this step may refer to, or be performed by, an input component as described with reference to FIG. 2.

At step 405, the system performs text analytics on the text corpus. In some cases, the operations of this step may refer to, or be performed by, an analytics component as described with reference to FIG. 2.

At step 410, the system determines if the patient has a need for transportation assistance based on the performed text analytics. In some cases, the operations of this step may refer to, or be performed by, a transportation assistance component as described with reference to FIG. 2.

At step 415, the system notifies a transportation need. In some cases, the operations of this step may refer to, or be performed by, a notification component as described with reference to FIG. 2.

At step 420, the system retrieves patient's appointment data from a structured data field or an unstructured source. In some cases, the operations of this step may refer to, or be performed by, an input component as described with reference to FIG. 2. In some examples, the appointment data may be retrieved from a structured source (e.g., a structured data field from a database, answers to form questions, etc.). In some examples, the appointment data may be retrieved from an unstructured source. The unstructured source can be speech, dictation, etc. stating, for example “patient has a job that has Tuesday and Thursday off”.

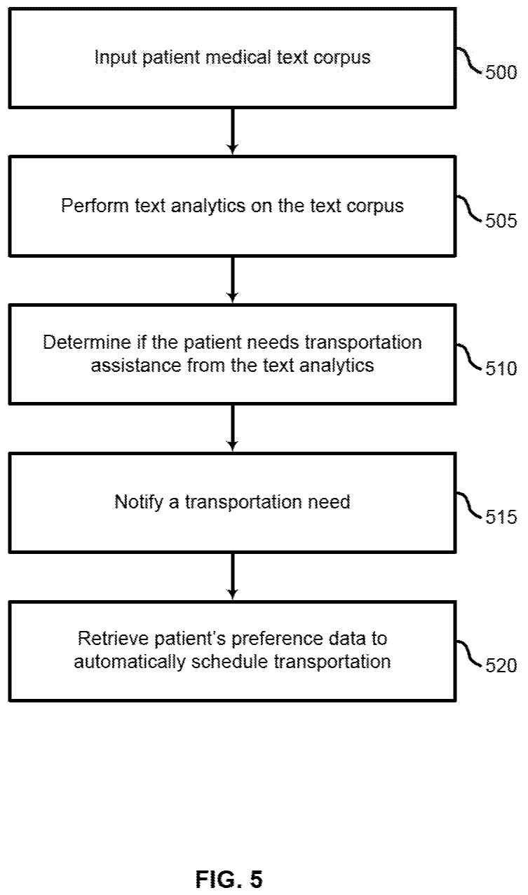

FIG. 5 shows an example of a process for notifying a transportation service based on text analytics in accordance with aspects of the present disclosure. In some examples, these operations may be performed by a system including a processor executing a set of codes to control functional elements of an apparatus. Additionally or alternatively, the processes may be performed using special-purpose hardware. Generally, these operations may be performed according to the methods and processes described in accordance with aspects of the present disclosure. For example, the operations may be composed of various substeps, or may be performed in conjunction with other operations described herein.

At step 500, the system inputs a text corpus comprising patient medical information. In some cases, the operations of this step may refer to, or be performed by, an input component as described with reference to FIG. 2.

At step 505, the system performs text analytics on the text corpus. In some cases, the operations of this step may refer to, or be performed by, an analytics component as described with reference to FIG. 2.

At step 510, the system determines if the patient has a need for transportation assistance based on the performed text analytics. In some cases, the operations of this step may refer to, or be performed by, a transportation assistance component as described with reference to FIG. 2.

At step 515, the system notifies a transportation need. In some cases, the operations of this step may refer to, or be performed by, a notification component as described with reference to FIG. 2.

At step 520, the system retrieves patient's preference data to automatically schedule transportation. In some cases, the operations of this step may refer to, or be performed by, an input component as described with reference to FIG. 2.

In some examples, transportation service may be automatically scheduled based on the patient's preference data. For instance, the patient may have a default preference of automatically scheduling transportation service. In that case, transportation service for the patient may be automatically scheduled. Alternatively, in other examples, the method of the present disclosure may send the patient an alert based on the patient's preference data. The alert can be a cellphone notification, an email, an automated voice call, etc. The alert may relate to inquiring if the patient wants transportation service to be scheduled. The patient may respond to the alert, and the response may be used to automatically schedule transportation service based on the patient's preference data.

Further, in some examples, patient availability information may be retrieved. The patient availability information may be used to automatically schedule transportation service based on the patient's preference data. For instance, if the patient is unavailable (e.g., canceled appointment, family emergency, etc.), transportation service may not be scheduled. If the patient is available, transportation service may be automatically scheduled based on the patient's preference data.

Additionally or alternatively, transport availability information of transportation service provider may be accessed. In some cases, the transport availability information may be used to automatically schedule transportation service based on the patient's preference data.

In some cases, the appointment data retrieved at step 420 may be used in scheduling transportation service. For example, the appointment data (e.g., appointment date, appointment time, etc.), individually or combined, may be used to schedule transportation service. In some cases, transportation service scheduling may occur via web service, REST API, etc.

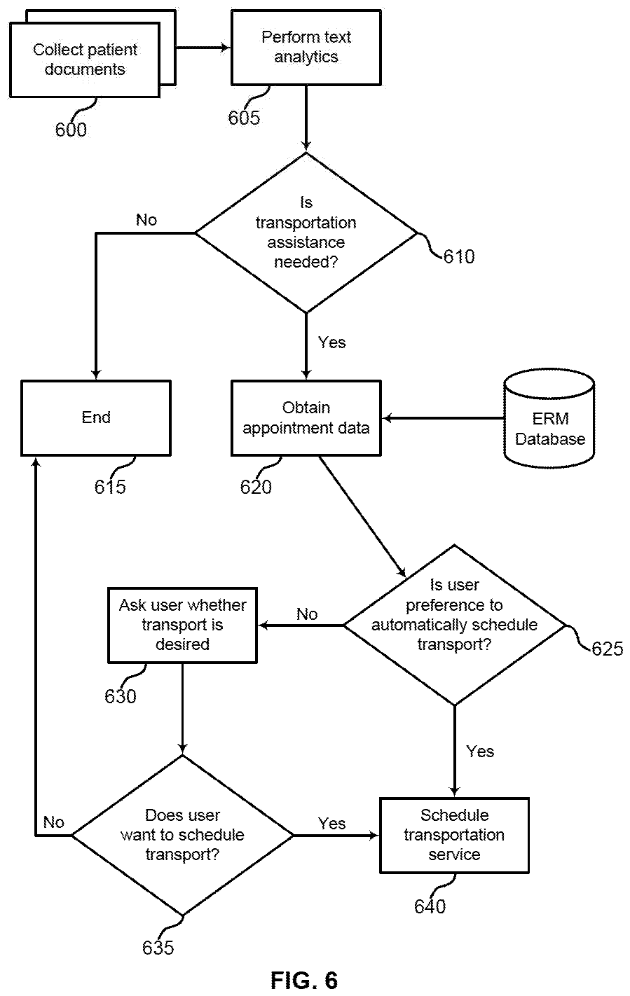

FIG. 6 shows another example of a process for notifying a transportation service based on text analytics in accordance with aspects of the present disclosure. In some examples, these operations may be performed by a system including a processor executing a set of codes to control functional elements of an apparatus. Additionally or alternatively, the processes may be performed using special-purpose hardware. Generally, these operations may be performed according to the methods and processes described in accordance with aspects of the present disclosure. For example, the operations may be composed of various substeps, or may be performed in conjunction with other operations described herein.

At step 600, the system may collect patient documents and information (i.e., documents related to patient medical records, or patient circumstances, or a patient's request for a medical appointment). In some cases, the patient information may include documents related to a previous patient visit, including audio files of conversations between the patient and medical professionals.

At step 605, the system may perform text analytics on the patient documents and information as described above.

At step 610, the system may determine whether transportation assistance is needed based on the text analytics. If assistance is need, the process may proceed to step 620. If not, the process may end at step 615.

At step 620, the system may obtain appointment data (e.g., based on information from an electronic medical records database). For example, the system may determine whether the patient is scheduled for an appointment with a specialist or a follow-up appointment.

At step 625, the system may determine whether the patient has a preference to be automatically scheduled for transportation assistance. If yes, the process may proceed to step 640. If not, the process may proceed to step 630.

At step 630, the system may alert the user and prompt the user regarding whether transportation assistance is desired in a particular instance.

At step 635, the system may determine whether the user wants to schedule transportation based on the prompt. If not, the process may end. If so, the process may proceed to step 640.

At step 640, the system may schedule transportation assistance for the medical appointment. In some cases, the type of transportation assistance scheduled may depend on factors such as time, location, and patient condition, etc.

Accordingly, the present disclosure includes the following embodiments.

In some examples, the text analytics is performed using NLP. In some examples, the NLP is performed using dictionary and rule based processing. In some examples, the NLP is performed using machine learning. In some examples, the machine learning is based on a Convolutional Neural Network (CNN), an Unsupervised Pretrained Network (UPN), a Recurrent Neural Network (RNN), a Long Short-Term Memory (LSTM) architecture, or a Recursive Neural Network.

Some examples of the method, apparatus, and non-transitory computer readable medium described above may further include retrieving patient's appointment data from a structured data field. Some examples of the method, apparatus, and non-transitory computer readable medium described above may further include retrieving patient's appointment data from an unstructured source.

Some examples of the method, apparatus, and non-transitory computer readable medium described above may further include retrieving patient's preference data to automatically schedule transportation. Some examples of the method, apparatus, and non-transitory computer readable medium described above may further include retrieving patient availability information. Some examples of the method, apparatus, and non-transitory computer readable medium described above may further include accessing transport availability data of a transportation service.

The description and drawings described herein represent example configurations and do not represent all the implementations within the scope of the claims. For example, the operations and steps may be rearranged, combined or otherwise modified. Also, structures and devices may be represented in the form of block diagrams to represent the relationship between components and avoid obscuring the described concepts. Similar components or features may have the same name but may have different reference numbers corresponding to different figures.

Some modifications to the disclosure may be readily apparent to those skilled in the art, and the principles defined herein may be applied to other variations without departing from the scope of the disclosure. Thus, the disclosure is not limited to the examples and designs described herein, but is to be accorded the broadest scope consistent with the principles and novel features disclosed herein.

The described methods may be implemented or performed by devices that include a general-purpose processor, a digital signal processor (DSP), an application specific integrated circuit (ASIC), a field programmable gate array (FPGA) or other programmable logic device, discrete gate or transistor logic, discrete hardware components, or any combination thereof. A general-purpose processor may be a microprocessor, a conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration). Thus, the functions described herein may be implemented in hardware or software and may be executed by a processor, firmware, or any combination thereof. If implemented in software executed by a processor, the functions may be stored in the form of instructions or code on a computer-readable medium.

Computer-readable media includes both non-transitory computer storage media and communication media including any medium that facilitates transfer of code or data. A non-transitory storage medium may be any available medium that can be accessed by a computer. For example, non-transitory computer-readable media can comprise random access memory (RAM), read-only memory (ROM), electrically erasable programmable read-only memory (EEPROM), compact disk (CD) or other optical disk storage, magnetic disk storage, or any other non-transitory medium for carrying or storing data or code.

Also, connecting components may be properly termed computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

In this disclosure and the following claims, the word “or” indicates an inclusive list such that, for example, the list of X, Y, or Z means X or Y or Z or XY or XZ or YZ or XYZ. Also the phrase “based on” is not used to represent a closed set of conditions. For example, a step that is described as “based on condition A” may be based on both condition A and condition B. In other words, the phrase “based on” shall be construed to mean “based at least in part on.”

What is claimed is:

1. A method for scheduling services, comprising:
inputting a text corpus comprising patient medical information;
performing text analytics on the text corpus;
determining if the patient has a need for transportation assistance based on the performed text analytics; and
notifying a transportation service of the need for transportation assistance based on the determination.

2. The method of claim 1, wherein:
the text analytics is performed using natural language processing (NLP).

3. The method of claim 2, wherein:
the NLP is performed using dictionary and rule based processing.

4. The method of claim 2, wherein:
the NLP is performed using machine learning.

5. The method of claim 4, wherein:
the machine learning is based on a Convolutional Neural Network (CNN), an Unsupervised Pretrained Network (UPN), a Recurrent Neural Network (RNN), a Long Short-Term Memory (LSTM) architecture, or a Recursive Neural Network.

6. The method of claim 1, further comprising:
retrieving patient's appointment data from a structured data field.

7. The method of claim 1, further comprising:
retrieving patient's appointment data from an unstructured source.

8. The method of claim 1, further comprising:
retrieving patient's preference data to automatically schedule transportation using the transportation service.

9. The method of claim 8, further comprising:
retrieving patient availability information.

10. The method of claim 8, further comprising:
accessing transport availability data of the transportation service.

11. An apparatus for scheduling services, comprising: a processor and a memory storing instructions and in electronic communication with the processor, the processor being configured to execute the instructions to:
perform natural language processing (NLP) on a text corpus comprising patient medical information;
determine if the patient has a need for transportation assistance based on the NLP; and
schedule a transportation service based on the determination.

12. The apparatus of claim 11, wherein:
the NLP is performed using dictionary and rule based processing.

13. The apparatus of claim 11, wherein:
the NLP is performed using machine learning.

14. The apparatus of claim 13, wherein:
the machine learning is based on a Convolutional Neural Network (CNN), an Unsupervised Pretrained Network (UPN), a Recurrent Neural Network (RNN), a Long Short-Term Memory (LSTM) architecture, or a Recursive Neural Network.

15. The apparatus of claim 11, further comprising:
retrieving patient's appointment data from a structured data field.

16. The apparatus of claim 11, further comprising:
retrieving patient's appointment data from an unstructured source.

17. The apparatus of claim 11, the processor being further configured to execute the instructions to:
retrieve patient's preference data to automatically schedule transportation.

18. The apparatus of claim 17, the processor being further configured to execute the instructions to:
retrieve patient availability information.

19. The apparatus of claim 17, the processor being further configured to execute the instructions to:
access transport availability data of the transportation service.

20. A non-transitory computer readable medium storing code for scheduling services, the code comprising instructions executable by a processor to:
perform text analytics on a text corpus;
retrieve patient's preference data;
determine if the patient has a need for transportation assistance based on the text analytics;
identify a transportation service of the need for transportation assistance based on the determination; and
automatically schedule transportation based on the identification and the patient preference data.
